(19) United States
(12) Patent Application Publication
(10) Pub. No.: US 2007/0003504 A1
(43) Pub. Date: Jan. 4, 2007

(54) Method of enhanced benzoyl peroxide application

(76) Inventors: Jose E. Ramirez, Trumbull, CT (US); Joseph R. Faryniarz, Middlebury, CT (US)

Correspondence Address: CARTER, DELUCA, FARRELL & SCHMIDT, LLP, 445 BROAD HOLLOW ROAD, SUITE 225, MELVILLE, NY, 11747, US

(21) Appl. No.: 11/476,959

(22) Filed: Jun. 28, 2006

Related U.S. Application Data
(60) Provisional application No. 60/695,033, filed on Jun. 29, 2005.

(51) Int. Cl. A61K 8/81; A61K 8/73; A61K 31/192

(52) U.S. Cl. 424/70.13; 424/70.16; 514/568

(57) ABSTRACT

Methods of treating acne are disclosed comprising applying a benzoyl peroxide solution in one or more solvents to the skin of a user and applying a sealer thereto. The methods are useful to improve the penetration of active drug into the skin in need thereof, and are highly effective in treating P. Acne.

### RELATED APPLICATION

This Application claims priority benefit of U.S. Provisional Application No. 60/695,033 filed Jun. 29, 2005, herein incorporated by reference in its entirety.

### BACKGROUND

1. Technical Field

This disclosure relates to application of benzoyl peroxide and, more specifically to a treatment regimen that improves percutaneous absorption of benzoyl peroxide by use of a sealer. The method generally has two steps including applying a suitable benzoyl peroxide composition to the skin, and applying a sealer to the treated skin. The method improves the penetration of the drug into the stratum corneum and the epidermis where it is most effective in treatment and the prevention of acne.

2. Background of Related Art

The integumentary system includes the skin and all the structures associated with skin such as hair, nails, sweat glands and oil glands. The functions of the integumentary system include, inter alia, providing a protective barrier for the body to prevent the entry of potentially harmful things. Unfortunately, the protective barrier function of the skin may slow and/or prevent the penetration of benzoyl peroxide applied to the skin and/or reduce the efficacy of topically applied benzoyl peroxide. Furthermore, during skin treatment, the problematic nature of the protective barrier function of the skin may be compounded by other factors such as characteristics of the benzoyl peroxide and excipients combined therewith during the formulation processes, the condition of the skin prior to application of the benzoyl peroxide, the amount of time the benzoyl peroxide remains in contact with the skin, and/or the diseased or injured state of the skin.

Although treatments are known for applying benzoyl peroxide, known treatments are problematic in that they do not demonstrate immediate penetration of benzoyl peroxide, and results may vary from patient to patient. Furthermore, no one treatment, if ever, obtains maximum benefit for every patient. As a result, some individuals may have slower benzoyl peroxide penetration and an increased risk for complications during healing. Accordingly, novel skin treatments are continuously sought after to help enhance penetration of benzoyl peroxide immediately upon application to skin and there remains room for improvement in skin treatment regimens to enhance benzoyl peroxide penetration.

What are needed are new skin care compositions and methods of applying benzoyl peroxide to skin that will actively enhance penetration of the benzoyl peroxide immediately upon application to skin; prevent removal of the benzoyl peroxide; hold the benzoyl peroxide in a reservoir film; and/or enhance long term penetration of benzoyl peroxide.

### SUMMARY

This disclosure relates to application of benzoyl peroxide and, more specifically to a treatment regimen that improves percutaneous absorption of benzoyl peroxide by use of a sealer. Applying a sealer to an area of skin treated with benzoyl peroxide will, among other things, actively enhance penetration of the benzoyl peroxide immediately upon application to skin, seal the skin surface to prevent removal of the benzoyl peroxide, hold the benzoyl peroxide in a reservoir film, and/or enhance long term penetration of the benzoyl peroxide. Accordingly, benzoyl peroxide such as topically applied benzoyl peroxide and formulations containing benzoyl peroxide may be used in combination with a sealer to treat undesirable skin conditions.

Skin having one or more undesirable skin conditions is treated in accordance with the present disclosure by topically treating skin with benzoyl peroxide, followed by the immediate application of a sealer to the treated skin. For example, compositions containing benzoyl peroxide can be directly applied to skin in need of treatment, prior to the application of a sealer. Post-treatment by application of a sealer, among other things, increases the penetration and/or efficacy of the benzoyl peroxide.

In addition, dermatological treatment regimens in accordance with the present disclosure may improve characteristics of a user's skin. The regimens include the repeated topical application of benzoyl peroxide, and the repeated topical application of a sealer.

These and other aspects of this disclosure will be evident upon reference to the following detailed description.

### DETAILED DESCRIPTION OF PREFERRED EMBODIMENTS

The present disclosure relates to compositions and methods for application of benzoyl peroxide to skin. The method includes applying a predetermined amount of benzoyl peroxide to an area of skin in need thereof and applying a sealer to the treated area. Sealer is applied over the benzoyl peroxide to an area of skin in need of treatment. In embodiments, the sealer dries on the skin surface sealing the remaining benzoyl peroxide in place. The application of the a sealer actively enhances penetration of the benzoyl peroxide immediately upon application to skin, seals the skin surface to prevent removal of the benzoyl peroxide, and/or enhances long term penetration of the benzoyl peroxide. In embodiments, the sealer holds benzoyl peroxide in a reservoir film.

In embodiments, a treatment regimen in accordance with this disclosure includes the sequential use of at least two products; namely at least one benzoyl peroxide composition and/or mixtures of formulations such as compositions containing benzoyl peroxide, and at least one sealer. In one embodiment, benzoyl peroxide may be combined with a blender composition. Here, the treatment includes the step of pre-mixing benzoyl peroxide composition with a blending composition to form a pre-mix; and then applying the pre-mix to the skin. In embodiments, the percutaneous absorption of the benzoyl peroxide in increased compared to the application of the benzoyl peroxide or pre-mix without a sealer.

Suitable benzoyl peroxide may be used either alone, or in combination with a composition and/or formulation. For example, the benzoyl peroxide may be in solution form, a topical formulation characterized as a clear facial serum, a topical formulation characterized as a stick, or an emulsion such as a blender composition.

In some embodiments, the present compositions provide a solvent vehicle formulation for the treatment of acne in which the major active ingredient is benzoyl peroxide. In embodiments, the active ingredients are provided in a previously unobtainable clear product forms. Such clear product forms include serums, toners, pump or aerosol sprays, clear gels, sticks, creams, lotions and mousses. The clear product forms improve the effectiveness of the ingredients applied, allowing the use of lower levels and providing quicker patient response. Also the use of these vehicle solvents in emulsion product forms allows levels of benzoyl peroxide in oil/water emulsions which exceed the solubility parameters of benzoyl peroxide in o/w systems without the solvent vehicle, resulting in a slurry type application with increased antimicrobial activity due to the solubilized fraction of the benzoyl peroxide. Also, the use of solvents that have relatively low water solubility and relatively high solubility for benzoyl peroxide are highly effective, and more effective than long chain alkyl benzoates in removing water from the crystals during processing, offering a safe handling method for other industrial uses.

In embodiments, compositions in accordance with the present disclosure include benzoyl peroxide in solution in one or more solvents. Benzoyl peroxide is normally commercially available as either pure (98% active) crystals or in a wet crystalline state containing 70 to 80% active benzoyl peroxide in 20-30% water. Any commercially available forms of benzoyl peroxide can be mixed with the disclosed solvents to form compositions in accordance with this disclosure.

The amount of benzoyl peroxide mixed with the solvent will vary depending on a number of factors, including, for example, the activity of benzoyl peroxide, the ultimate form of the product and the particular disclosed solvent employed. Generally, the benzoyl peroxide will constitute an amount of 1 to 70 weight percent of the benzoyl peroxide/solvent mixture. In embodiments, the benzoyl peroxide constitutes an amount of about 3.00 to about 40 weight percent of the benzoyl peroxide/solvent mixture. In embodiments, the benzoyl peroxide constitutes an amount of about 2 to about 15 weight percent of the benzoyl peroxide/solvent mixture. In embodiments, benzoyl peroxide is present in amounts effective for treating Acne Vulgaris. Due to the increased availability of the benzoyl peroxide in the present compositions, the minimum effective amount of benzoyl peroxide may, in embodiments, be lower than the amounts in presently available acne treatments containing benzoyl peroxide. Thus, compositions in accordance with the present disclosure may contain 5% benzoyl peroxide and have 2 to 3 times the skin penetration than conventional 10% benzoyl peroxide compositions.

Solvents useful for preparing the present compositions such as solutions include any solvent capable solubilizing benzoyl peroxide. Non-limiting examples of such solvents include short chain alkyl esters, ethers, aldehydes, ketones or alcohols of benzoic acid, benzyl alcohol, salicylic acid, phenol or phathalic acid. As used herein “short chain” refers to molecules having two to six carbon atoms (C2-C6). Other suitable solvents include aryl esters, ethers, aldehydes, ketones and alcohols of benzoic acid, benzyl alcohol, salicylic acid, phenol and phthalic acid. In certain embodiments, the compositions in accordance with the present disclosure include one or more of the following classes of solvent: alkyl esters of benzoic acid, alkyl esters of benzyl alcohol, alkyl esters of salicylic acid, alkyl esters of phenol, alkyl esters of phthalic acid, alkyl ethers of benzyl alcohol, alkyl esters of phthalic acid, alkyl ethers of benzyl alcohol, alkyl ethers of phenol. Additional non-limiting examples of suitable solvents include benzoyl benzoate, benzoyl alcohol, diethyl phthalate, benzoic acid 2-phenyl ethyl ester, methyl salicylate, ethyl salicylate, propyl salicylate, butyl salicylate, ethyl benzoate, methyl benzoate, propyl benzoate, butyl benzoate, dimethyl phthalate, diethyl phthalate, benzyl ethyl ether, benzyl methyl ether, phenetole, phenyl acetone, phenyl ethyl alcohol, phenoxyethanol, phenyl acetaldehyde, ethyl phenyl acetate, phenyl methyl ketone, phenyl acetate, benzyl acetate, benzyl aceto acetate, benzyl formate, benzaldehyde, benzyl alcohol, ethyl benzyl alcohol, salicylaldehyde, benzyl salicylate, phenyl tolyl ketone, phenyl benzoate, phenyl ether, dibenzyl ether, benzyl benzoate, benzoic acid and 2-phenyl ethyl ester.

The amount of solvent mixed with the benzoyl peroxide will vary depending on a number of factors, including, for example, the ultimate form of the product and the particular solvent employed. Generally, the solvent will constitute an amount of about 1 to 70 weight percent of the benzoyl peroxide/solvent mixture. In embodiments, the solvent constitutes an amount of about 10 to about 50 weight percent of the total composition. In embodiments, the solvent is present in an amount of about 20 to about 40 weight percent of the total composition. In embodiments, solvent is present in amounts effective for dissolving benzoyl peroxide.

In addition to the solvent in which benzoyl peroxide is soluble, the compositions in accordance with the present disclosure may contain one or more secondary solvents. Suitable secondary solvents include, for example, ethanol, acetone, dimethyl isosorbide, and glycol ethers of C₁ to C₆ alcohols with no greater than 2 moles of ethylene oxide. Suitable glycol ethers include glycol ethers of phenol with no greater than 2 moles of ethylene oxide, glycol ethers of methanol with no greater than 2 moles of ethylene oxide, glycol ethers of ethanol with no greater than 2 moles of ethylene oxide and glycol ethers of propanol with no greater than 2 moles of ethylene oxide. Non-limiting examples of such co-solvents include phenoxy ethanol, ethoxy diglycol and propylene glycol methyl ether.

The amount of secondary solvent mixed with the benzoyl peroxide/solvent mixture will vary depending on a number of factors, including, for example, the ultimate form of the product and the particular solvent and/or secondary solvent employed. Generally, the secondary solvent is present in an amount of about 1 to 40 weight percent of the total composition. In embodiments, the secondary solvent is present in an amount of about 5 to about 30 weight percent of the total composition. In embodiments, the secondary solvent is present in an amount of about 10 to about 20 weight percent of the total composition.

In preparing some compositions in accordance with this disclosure, benzoyl peroxide is simply mixed with the disclosed solvents, which may occur room temperature. As previously mentioned, benzoyl peroxide is normally commercially available as either pure crystals or in a wet crystalline state. Any of these or other forms of benzoyl peroxide can be mixed with the disclosed solvents to form compositions in accordance with this disclosure.

In addition to benzoyl peroxide, the present compositions may also optionally include salicylic acid, antibiotics and/or any other material in amounts effective for acne treatment. Antimicrobials which may be combined with benzoyl peroxide compositions in accordance with the present disclosure include all antibiotics, antimicrobial agents and antimicrobial peptides. Non-limiting examples of suitable antibiotics include inter alia dermatologically acceptable salts of tetracylin and tetracyclin derivatives, gentamycin, kanamycin, streptomycin, neomycin, capreomycin, lineomycin, paromomycin, tobramycin, erythromycin, triclosan, octopirox, parachlorometa xylenol nystatin, tolnaftate, miconazole hydrochloride, chlorhexidine gluconate, chlorhexidin hydrochloride, methanamine hippurate, methanamine mandelate, minocycline hydrochloride, clindamycin, cleocin, b-lactam derivatives such as aminopenicillin and mixtures thereof. In embodiments, a combination of chlorhexidin gluconate and triclosan is suitable for use herein. In embodiments antimicrobial agents that may be used in accordance with the present disclosure either alone or in combination include for example benzoyl peroxide and salicylic acid.

The amount of antibiotic mixed with the benzoyl peroxide/solvent mixture will vary depending on a number of factors, including, for example, the ultimate form of the product and the particular solvent and/or secondary solvents employed. Generally, the antimicrobial will be present in an amount of about 1.0 to 30 weight percent of the total composition. In embodiments, the antibiotic is present in an amount of about 0.1 to about 5 weight percent of the total composition.

In embodiments, benzoyl peroxide can be added to the solvents to form a mixture at room temperature, e.g., at a temperature of 25 to about 27° C. Additionally, the benzoyl peroxide/solvent mixture also can be added to other ingredients to form desired products, e.g., emulsions, lotions, creams or gels at low temperatures. In these processes, since benzoyl peroxide is never in contact with substantial heat, the possibility of decomposition or fire is greatly reduced. However the present compositions include benzoyl peroxide in solution at levels as high as 10% by weight of the total formulation. These solutions can offer new clear products of increased efficacy.

Furthermore, the higher affinity of benzoyl peroxide to the disclosed solvents offers improved method for preparing anhydrous benzoyl peroxide without subjecting the composition to any heat during processing. For example, when benzoyl peroxide-wet crystals containing 25% water are mixed with solvents in accordance with the present disclosure, the solvents (which solubilize the benzoyl peroxide) replace water in the process of changing the crystalline benzoyl peroxide into a solution, water can readily be separated. Secondly, if levels of benzoyl peroxide are desired that exceed the solubility parameters of the solvents a saturated solution in conjunction with a fine soft benzoyl peroxide slurry is formed. This composition can then be filtered to remove the water from the composition, thereby providing a fine textured, substantially, water reduced benzoyl peroxide paste/saturated solution composition. The benzoyl peroxide solutions offer a way to use benzoyl peroxide in other industrial applications where anhydrous solutions will offer advantages over dispersions. In addition, the present process provides the advantage of more effective water removal if required for the desired application.

In embodiments thickeners and/or rheology modifiers such as fumed silica may be added to the solutions to increase the viscosity of the compositions and/or gel the compositions. In embodiments, the thickener and/or rheology modifiers may be present in an amount of about 0.1 to about 10 weight percent of the total composition. As mentioned above, the benzoyl peroxide corrective compositions in accordance with the present disclosure can be added to product forms.

The benzoyl peroxide, benzoyl peroxide solutions and/or benzoyl peroxide compositions may be combined with numerous ingredients to form products to be applied to the skin, or other tissues of humans or other mammals. Such products may include a dermatologically or pharmaceutically acceptable carrier or diluent, vehicle or medium, for example, a carrier, vehicle or medium that is compatible with the tissues to which they will be applied. The term “dermatologically or pharmaceutically acceptable” as used herein, means that the compositions or components thereof so described are suitable for use in contact with these tissues or for use in patients in general without undue toxicity, incompatibility, instability, allergic response, and the like. In embodiments, compositions in accordance with the present disclosure can contain any ingredient conventionally used in cosmetics and/or dermatology.

As an illustrative example, compositions can be formulated to contain benzoyl peroxide in an amount of about 0.001 to about 20% by weight of the total composition. In embodiments, products can be formulated to contain benzoyl peroxide in an amount of about 0.05 to about 10% by weight of the total composition. In other embodiments, the amount of benzoyl peroxide is present in an amount of about 0.1 to about 5.0% by weight of the total composition. In such embodiments, the benzoyl peroxide present may be in a pharmaceutically acceptable salt form.

In embodiments, products containing benzoyl peroxide in accordance with the present disclosure can be in the form of solutions, emulsions (including microemulsions), suspensions, creams, fluid cream, oils, lotions, gels, powders, sticks, or other typical solid or liquid compositions used for treatment of undesirable skin conditions. Such compositions may contain, in addition to the benzoyl peroxide and/or benzoyl peroxide compositions in accordance with this disclosure, other ingredients typically used in such products, such as other active cosmetic substances such as retinol, retinol derivatives, allantoin, tocopherol, tocopherol derivatives, niacinamide, phytosterols, isoflavones, panthenol, panthenol derivatives, bisabolol, farnesol, and combinations thereof, other active drug substances such as corticosteroid, metronidazole, sulfacetamide, sulfur, and combinations thereof, antioxidants, antimicrobials, coloring agents, detergents, dyestuffs, emulsifiers, emulsifying wax, emollients, fillers, fragrances, gelling agents, hydration agents, moisturizers, odor absorbers, natural or synthetic oils, penetration agents, powders, preservatives, solvents, surfactants, thickeners, viscosity-controlling agents, water, distilled water, waxes, and optionally including anesthetics, anti-itch actives, botanical extracts, conditioning agents, darkening or lightening agents, glitter, humectant, mica, minerals, polyphenols, phytomedicinals, silicones or derivatives thereof, skin protectants, sunblocks, vitamins, and mixtures or combinations thereof. Such compositions may also contain, in addition to the benzoyl peroxide and/or benzoyl peroxide compositions in accordance with this disclosure, one or more: fatty alcohols, fatty acids, organic bases, inorganic bases, wax esters, steroid alcohols, triglyceride esters, phospholipids, polyhydric alcohol esters, fatty alcohol ethers, hydrophilic lanolin derivatives, hydrophilic beeswax derivatives, cocoa butter waxes, silicon oils, pH balancers, cellulose derivatives, hydrocarbon oils, or mixtures and combinations thereof.

In embodiments, product forms can be formulated to contain humectant in amounts from about 1% to about 15% by weight of the total composition. For example glycerine can be added to the composition in amounts from about 1% to about 15% by weight of the total composition. In particular embodiments, glycerine can be added to the composition in amounts from about 1% to about 5% by weight of the total composition.

In embodiments, product forms can be formulated to contain solvent in an amount of about 1% to about 45% by weight of the total composition. For example petroleum derivatives such as propylene glycol can be added to the composition in an amount of about 1% to about 45% by weight of the total composition. In particular embodiments, propylene glycol, polyethylene glycol, ethoxy diglycol can be added to the composition in an amount of about 15% to about 30% by weight of the total composition.

In embodiments, product forms can be formulated to contain water in an amount of about 40% to about 99% by weight of the total composition. For example distilled water can be added to the composition in an amount of about 40% to about 99% by weight of the total composition. In particular embodiments, distilled water can be added to the composition in an amount of about 65% to about 80% by weight of the total composition.

In embodiments, benzoyl peroxide compositions in accordance with the present disclosure are useful in the formation of oil-in-water emulsion product forms. Conventional emulsion formulation typically requires mixing the aqueous phase ingredients and the dispersant with heating until a uniform solution or dispersion is obtained (optionally in several stages), mixing the organic phase ingredients with heating until a uniform solution or dispersion is obtained (also optionally in several stages), then adding the aqueous phase to the organic phase with agitation (e.g. stirring or other shearing or heating technique) to form an oil-in-water emulsion of the two phases. However, heating steps are problematic in that heat decomposes organic peroxides such as benzoyl peroxide. In embodiments, emulsion compositions in accordance with the present disclosure are capable of a low temperature blending and shearing techniques that do not require an intensive heating step of 70° C. or above. Accordingly, such blending can occur at room temperature.

In some emulsion embodiments, the aqueous phase constituting the dispersion medium may include any suitable surfactant, humectant, suspending agent, and/or buffer systems, and combinations thereof suitable for combining with benzoyl peroxide.

Non-limiting examples of suitable surfactants include natural compounds, such as phospholipids and cholates, or nonnatural compounds such as: polysorbates, which are fatty acid esters of polyethoxylated sorbitol; polyethylene glycol esters of fatty acids from sources such as castor oil; polyethoxylated fatty acid, e.g. stearic acid; octylphenolpoly(ethyleneglycolether); polyethoxylated isooctylphenol/formaldehyde polymer; poloxamers, e.g., poly(oxyethylene)poly(oxypropylene) block copolymers; polyoxyethylene fatty alcohol ethers; polyoxyethylene nonylphenyl ethers; polyoxyethylene isooctylphenyl ethers; SDS, and combinations thereof.

In embodiments, non-limiting examples of suitable mixtures of surfactant molecules, including mixtures of surfactants of different chemical types, are acceptable. Surfactants should be suitable for cosmetic or pharmaceutical administration and compatible with the benzoyl peroxide to be delivered. Non-limiting examples of surfactants include phospholipids such as phosphatidylcholines (lecithins), including soy or egg lecithin. Other suitable phospholipids include phosphatidylglycerol, phosphatidylinositol, phosphatidylserine, phosphatidic acid, cardiolipin, and phosphatidylethanolamine. The phospholipids may be isolated from natural sources or prepared by synthesis.

Non-limiting examples of suitable suspending agents include the following constituents: polyacrylamide, C13-14 isoparafin & laureth 7; C13-14 isoparaffin, mineral oil, polyacrylate, polyacrylamide and ethoxylated sorbitan ester; acrylamide/sodium acryloyidimethyl taurate copolymer, isohexadecane and ethoxylated sorbitan ester; and combinations thereof. However any cosmetically or pharmaceutically acceptable suspending agent suitable for combining with benzoyl peroxide may be used.

Non-limiting examples of suitable humectants include glycerin; however any material capable of obtaining moisture may be added provided it is stable with benzoyl peroxide.

The products formulated with the present solutions can be packaged in any type of container within the purview of those skilled in the art, including, but not limited to bottles, tubes, pump type, roll-ons, daubers, wipes, and the like.

The treatments in accordance with the present disclosure also include applying a sealer to the area of skin in need thereof. The application of the sealer is useful in actively enhancing penetration of the benzoyl peroxide immediately upon application to skin, sealing the skin surface to prevent removal of the benzoyl peroxide, benzoyl peroxide solution, benzoyl peroxide composition and/or benzoyl peroxide formulation holding them in a reservoir film, and/or enhancing long term penetration of the drug or benzoyl peroxide composition and/or benzoyl peroxide formulation.

The sealer may be made of a number of constituents including one or more solvents, penetration aids, and polymer substances including film-forming substances, and combinations thereof.

Non-limiting examples of suitable polymers for use in accordance with the sealer of the present disclosure include natural polymers, acrylic resins, silicones, celluloses, alkyd resins, carboxyvinyl polymers, vinylpyrrolidone-based polymers, type A methacrylic acid copolymer such as Eudragit L 100 brand copolymer, type B methacrylic acid copolymer such as Eudragit S 100 brand copolymer, and combinations thereof.

Non-limiting examples of suitable acrylic resins for use in accordance with the sealer of the present disclosure include polyacrylic acid, poly(methyl acrylate), poly-(ethyl acrylate), poly(butyl acrylate), polyacrylamide, poly(N-isopropylacrylamide), ammonium polyacrylate, sodium polyacrylate), crosslinked sodium polyacrylate, polymethacrylic acid, poly(methyl methacrylate, poly-(ethyl methacrylate), poly(butyl methacrylate), polymethacrylamide, sodium methacrylate, acrylic acid-styrene-ammonium methacrylate copolymers, acrylic acid-styrene copolymers, acrylic acid-methacrylamide copolymers, alkyl acrylate-styrene copolymers, alkyl acrylate copolymers, ethyl acrylate-acrylamide-acrylic acid copolymers, ethyl acrylate-butyl acrylate copolymers, ethyl acrylate-ethyl methacrylate copolymers, ethyl acrylate-methyl methacrylate-acrylic acid copolymers, ethyl acrylate-methyl methacrylate copolymers, ethyl acrylate-methacrylic acid copolymers, octyl acrylate-styrene copolymers, octyl acrylate-vinyl acetate copolymers, hydroxypropyl acrylate-butylaminoethyl methacrylate-acrylic acid acrylamide copolymers, butyl acrylate-ethyl hydroxymethacrylate copolymers, butyl acrylate-hydroxymethacrylic acid copolymers, butyl acrylate-methyl methacrylate copolymers, butyl acrylate-methacrylic acid copolymers, butyl acrylate-vinyl acetate copolymers; methyl acrylate-ethyl acrylate copolymers, methyl acrylate-styrene copolymers, methoxyethyl acrylate-hydroxyethyl acrylate-butyl acrylate copolymers, methoxyethyl acrylate-hydroxyethyl acrylate copolymers, acrylic resin alkanolamines, methacrylic acid-styrene copolymers, methacrylic acid-butyl methacrylate copolymers, methacrylic acid-methyl methacrylate copolymers, methyl methacrylate-butyl acrylate-octyl acrylate copolymers, and the like, and combinations thereof.

Non-limiting examples of suitable celluloses for use in accordance with the sealer of the present disclosure include film-forming polymer, methyl cellulose, ethyl cellulose, cationized cellulose, carboxymethyl cellulose, hydroxyethyl cellulose, hydroxypropyl cellulose, hydroxypropylmethyl cellulose, etc., and combinations thereof.

Other non-limiting examples of suitable polymers for use in the sealer in accordance with the present disclosure include poly(vinyl methyl ether), vinyl methyl ether-ethyl maleate copolymers, vinyl methyl ether-butyl maleate copolymers, styrene-methylstyrene-indene copolymers, toluenesulfonamide resins, polyamide epichlorohydrin, polyethylene-imine, polyethylene glycol-epichlorohyd rin-coconut oil alkylamine-dipropylenetriamine condensates, polyvinyl acetal diester aminoacetate, polyvinyl acetal diethylamino-acetate, poly(dimethylmethylenepieridinium chloride), methoxyethylene-maleic anhydride copolymers, dimethyidiallylammonium chloride-acrylamide copolymers, hydrogenated styrene-methylstyrene-indene copolymers, maleic anhydride-diisobutylene copolymer sodium salts, polyvinyl alcohol, polyvinyl butyrate, polyvinyl chloride, vinyl acetate-crotonic acid copolymers, vinyl acetate-styrene copolymers, butadiene-acrylonitrile copolymers, and the like, and combinations thereof.

In embodiments, polymer such as a film-forming polymer is added in an amount of about 3% to about 30% of the total weight of the sealer composition. In some embodiments, at least one film-forming polymer includes polyacrylic acid and/or methacrylic acid copolymer in an amount of about 5% to about 20% of the total weight of the sealer.

Solvents useful for preparing sealer solutions include any solvent capable of solubilizing suitable polymers including film-forming polymers. Such solvents include solvents capable of solubilizing natural polymers, acrylic resins, silicones, celluloses, alkyd resins, carboxyvinyl polymers, vinylpyrrolidone-based polymers, methacrylic acid copolymer, and combinations thereof. In some embodiments, such solvents include water, short chain alkyl esters, ethers, aldehydes, ketones, alcohols, and combinations thereof. In one embodiment, the sealer includes a low molecular weight solvent system of acetone and ethanol for solubilizing the film-forming polymer. In embodiments, solvents suitable for solvating anionic copolymers based on methacrylic acid and methyl methacrylic are suitable for use in accordance herein including methanol, ethanol, aqueous isopropyl alcohol, acetone, and combinations thereof.

In embodiments, solvent is added in an amount of about 30% to about 96% of the total weight of the sealer. In some embodiments, solvent includes acetone and ethanol in an amount of about 50% to about 90% of the total weight of the sealer.

Penetration aids suitable for use in the sealer in accordance with the present disclosure include any penetration aid capable of disruption the barrier function of the skin. Non-limiting examples of suitable penetration aids include enzymes, keratolytic agents, acids, surfactants, DMSO, 1-dodecylazacycloheptan-2-one (available as Azone from the Upjohn Co.) and the like, 2-hydroxybenzoic acid, and combinations thereof. Penetration aids may be added to the sealer in an effective amount to disrupt the barrier function of the skin. In embodiments, the sealer solution may include penetration aid in the amount of about 5% to about 20% by weight of the total composition. Suitable amounts of salicylic acid or 2-hydroxybenzoic acid include an amount of about 10% to about 20% by weight of the total formulation.

In preparing sealer compositions in accordance with this disclosure, film-forming agent and/or polymer is simply mixed with the disclosed solvents, at room temperature or other suitable temperature. In embodiments, at least one penetration aids is added to the sealer mix. In embodiments, the sealer is characterized as a solvent based polymer solution.

In some embodiments, sealer may be applied in a predetermined amount to skin having a predetermined amount of benzoyl peroxide previously applied thereto. In embodiments, the sealer dries on the skin surface of the skin forming a film which seals the remaining drug in place. Benzoyl peroxide and compositions thereof trapped under this film will not be readily removed from the skin surface and therefore can still be absorbed. Another benefit is that this film acts like a bandage to protect skin surface from further environmental infections.

In some embodiments, the sealer may be any suitable film for covering a treated area on human skin. One useful embodiment contains sealer having constituents selected from the following: polyacrylic acid, methacrylic acid copolymer, salicylic acid, ethanol, methanol, isopropyl alcohol, acetone, cellulose ether, hydroxypropylcellulose, and combinations thereof.

In embodiments, the sealer may further include at least one solvent, at least one film-forming polymer, and combinations thereof. In some embodiments, the at least one solvent includes acetone and ethanol in an amount of about 50% to about 90% of the total weight of the sealer. In some embodiments, the at least one solvent includes acetone and/or ethanol in an amount of about 50% to about 90% of the total weight of the sealer. In embodiments, the at least one film-forming polymer comprises polyacrylic acid and/or methacrylic acid copolymer in an amount of about 5% to about 20% of the total weight of the sealer.

During treatment in accordance with the present disclosure benzoyl peroxide, benzoyl peroxide compositions and/or formulations or products containing benzoyl peroxide may be topically applied to skin in need of improvement in amounts sufficient to reduce or eliminate undesirable skin conditions. As used herein the word “treat,” “treating” or “treatment” refers to using the benzoyl peroxide, benzoyl peroxide compositions and/or formulations of the present disclosure prophylactically to prevent outbreaks of any undesirable skin condition such as acne Vulgaris, and/or therapeutically to ameliorate or cure an existing undesirable skin condition such as acne Vulgaris. A number of different treatments are now possible, which reduce and/or eliminate undesirable skin conditions. In some embodiments, benzoyl peroxide, benzoyl peroxide compositions and/or formulations of the present disclosure are used for cosmetic purposes only.

As used herein “skin condition” refers to any detectable skin manifestations caused by one or more pathogens or microbes. Such manifestations can be compounded due to a number of factors such as, for example, chronological aging, environmental damage, and/or other diseased or dysfunctional state. Non-limiting examples of such manifestations include the development of skin lines, crevices, bumps, comedones, craters, scaliness, flakiness and/or other forms of skin unevenness, roughness, or mottled appearance. It is understood, that the listed skin conditions are non-limiting and that only a portion of the skin conditions suitable for treatment in accordance with the present disclosure are listed herein.

In embodiments, compositions for use in accordance with the present disclosure contain benzoyl peroxide in an effective amount to improve undesirable skin conditions. As used herein “effective amount” refers to an amount of a compound or composition having benzoyl peroxide in accordance with the present disclosure that is sufficient to induce a particular positive benefit to skin having a skin condition. The positive benefit can be health-related, or it may be more cosmetic in nature, or it may be a combination of the two. In embodiments, the positive benefit is achieved by contacting skin with a combination of solvated benzoyl peroxide, and/or one or more antibiotic constituents, to improve a skin condition such as Acne Vulgaris.

The particular benzoyl peroxide concentration in the compositions generally depends on the purpose for which the composition is to be applied. For example, the dosage and frequency of application can vary depending upon the type and severity of the skin condition such as acne Vulgaris.

Treatments in accordance with the present disclosure contact skin with benzoyl peroxide in an effective amount to improve acne related skin conditions, followed by the application of a sealer in accordance with the present disclosure. In embodiments, patients are treated by topically applying to skin suffering from an acne related condition, one or more benzoyl peroxide compositions. The active ingredient is applied until the treatment goals are obtained. However, the duration of the treatment can vary depending on the severity of the condition. For example, treatments can last several weeks to months depending on whether the goal of treatment is to reduce or eliminate an acne related skin condition. In embodiments, sealer may include a penetration aid to further promote absorption of the benzoyl peroxide.

Treatments in accordance with the present disclosure increase the percutaneous absorption of benzoyl peroxide by contacting skin with an effective amount of one or more benzoyl peroxide composition in accordance with the present disclosure, followed by the application of a sealer in accordance with the present disclosure. In embodiments, subjects are treated with dissolved benzoyl peroxide by topically applying the dissolved mixture to skin. The benzoyl peroxide may be applied until the absorption goals are obtained. In embodiments, the percutaneous absorption of the benzoyl peroxide is increased compared to application of the undissolved benzoyl peroxide compositions. Accordingly, higher concentrations of benzoyl peroxide can be found in the epidermis, stratum corneum, and stratum corneum surface in a single application, than when compared to formulations utilizing undissolved benzoyl peroxide.

In some embodiments, the present method of treatment provides compositions and methods for treating acne. The system includes applying a predetermined amount of the active drug benzoyl peroxide in solution form to an area of skin in need thereof and applying a sealer to the treated area. The solvated benzoyl peroxide molecules are available to penetrate into follicles, follicle oil glands, the stratum comeum and epidermis of skin in need of treatment, including infected areas. Benzoyl peroxide saturates the targeted infected areas to destroy the P. Acne bacteria. Since, in some embodiments, the penetrated benzoyl peroxide is in solution, it is available to skin in need thereof in the most bio-effective form. One or more sealer is applied over the benzoyl peroxide solution to an area of skin in need of treatment or prophylaxis. In embodiments, the sealer is characterized as a film which may dry on the skin surface sealing the remaining drug in place. Drug trapped under this film will not be readily removed from the skin surface and therefore can still be absorbed.

In some embodiments, the treatments in accordance with the present disclosure can be combined with other treatments which pre-condition and/or post-condition skin in need of treatment. For example, in order to prepare the skin, a cleanser and/or toner can be applied to the treated area prior to the application of active agent and sealer.

In embodiments, the active agents and sealers are applied for cosmetic purposes only.

In some embodiments, use of a penetration aid such as 2-hydroxybenzoic acid may be included in the manufacture of a sealer for treatment of a skin condition such as acne Vulgaris. In such embodiments, penetration aids described in accordance with the present disclosure can be manufactured into sealer compositions. Such sealer compositions may also contain medicament, and/or formulations, and/or any excipients or ingredients described herein.

In order that those skilled in the art may be better able to practice the compositions and methods described herein, the following examples are given as an illustration of the preparation of the present compositions and methods. It should be noted that the disclosure is not limited to the specific details embodied in the examples.

### EXAMPLES 1-6

Non-limiting examples of clear serums suitable for use in accordance with the present disclosure were formulated having the following compositions:

| | EXAMPLES # | | | | | |
|---|---|---|---|---|---|---|
| | 1 | 2 | 3 | 4 | 5 | 6 |
| Dimethyl Isosorbide | 21.6% | 21.6% | 21.6% | 25.0% | 25.0% | 25.0% |
| Benzoic acid, 2-phenyl ethanol ester | 21.6% | 21.6% | 21.6% | 24.0% | 26.0% | 24.0% |
| Benzyl Peroxide wet with 26% water | 3.14% | 3.14% | 3.14% | 3.14% | 3.14% | 3.14% |
| Benzoic acid | 5.0% | 5.0% | 5.0% | 5.0% | 5.0% | 5.0% |
| Salicyclic acid | 2.0% | 2.0% | 2.0% | 2.0% | 2.0% | 2.0% |
| Benzyl Alcohol | 21.66% | 21.66% | 21.66% | 25.86% | 25.86% | 25.86% |
| Propylene glycol monomethyl ether | 25.0% | — | — | 15.0% | — | — |
| Ethanol | — | — | 25.0% | — | — | 15.0% |

These compositions were prepared by adding benzoyl peroxide to the solvents to form a mixture at low temperatures. Then the other ingredients were added with continued mixing to provide a clear cosmetic product.

### EXAMPLES 7-18

Non-limiting examples of serums suitable for use as actives in accordance with the present disclosure were formulated having the following constituents shown in percent weight of the total composition:

| | Example # | | | | | | | | | | | |
|---|---|---|---|---|---|---|---|---|---|---|---|---|
| | 7 | 8 | 9 | 10 | 11 | 12 | 13 | 14 | 15 | 16 | 17 | 18 |
| Benzyl Peroxide | 10 | 10 | 10 | 10 | 8 | 8 | 8 | 8 | 8 | 8 | 8 | 6.25 |
| 98% | | | | | | | | | | | | |
| Dimethyl | | | 30 | 40 | 46 | 43.5 | 45 | 45 | 45 | 45 | 45 | 40 |
| Isosorbide | | | | | | | | | | | | |
| Benzyl Benzoate | 85 | | 60 | | | | | | 18.5 | | | 42.45 |
| Acetone | 5 | 5 | | | | | | | | | | |
| Benzoic acid, 2- | | 85 | | 30 | 46 | 43.5 | 23.5 | 18.5 | — | 37 | 34 | |
| phenyl ethyl ester | | | | | | | | | | | | |
| Benzyl Alcohol | | | | 20 | | | 23.5 | 18.5 | 18.5 | | 3 | |
| Ethoxy ethanol | | | | | | 5 | | 10 | 10 | 10 | 10 | |
| Vitamin E Acetate | | | | | | | | | | | | 0.5 |
| BHT | | | | | | | | | | | | 0.8 |
| Ethoxy diglycol | | | | | | | | | | | | 10 |

These compositions were prepared by adding benzoyl peroxide to the solvents to form a mixture at room temperature. Then the other ingredients were added with continued mixing to provide a clear serum.

### EXAMPLE 19

Non-limiting benzoyl peroxide compositions for use in accordance with the present disclosure may be formulated having the following constituents shown in percent weight of the total composition:

| Ingredient | Amount (RANGE) |
|---|---|
| Benzoyl Peroxide | 4.0-6.25% |
| Benzoyl benzoate | 35-50% |
| Dimethyl isosorbide | 30-50% |
| BHT | 0.2-5% |
| Ethoxy diglycol | 5-20% |
| volatile silicone | 0-20% |
| Fumed silica | 0-10% |

### EXAMPLES 20

A non-limiting emulsion formulation suitable for use in accordance with the present disclosure was formulated having the following composition:

| Ingredient | Amount |
|---|---|
| Water | 60-80% |
| Steareth 40 | 0.1-5% |
| Glycerine | 1-5% |
| Benzoyl Peroxide 74% | 5-10% |
| Benzyl benzoate | 5-15% |
| Cyclomethicone | 1-10% |
| Ethylene Diamine tetraacetic acid | 0.1-3% |
| disodium salt (EDTA) | |
| Stearyl alcohol | 1-5% |
| Streareth 2 | 1-5% |

### EXAMPLE 21

A non-limiting example of a benzoyl peroxide emulsion suitable for use in accordance with the present disclosure includes:

| | Amount |
|---|---|
| Phase A Ingredients | |
| Benzoyl Peroxide 75% wet with water | 8.68% |
| Benzyl Benzoate | 10.00% |
| BHT | 0.4% |
| Vitamin E Acetate | 0.5% |
| Dimethyl Isosorbide | 3.00% |
| Phase B Ingredients | |
| DI Water | 74.22% |
| Phenoxyethanol | 0.1% |
| EDTA disodium salt | 0.1% |
| Hydroxyethylacrylate/sodium acryloyldimethyltaurate | 3.0% |
| copolymer & squalane & polysorbate 60 | |

### EXAMPLE 22

Another non-limiting emulsion formulation suitable for use in accordance with the present disclosure includes:

| | Amount |
|---|---|
| Phase A Ingredients | |
| Benzoyl Peroxide 75% wet with water | 8.68% |
| Benzyl Benzoate | 10.50% |
| BHT | 0.4% |
| Dimethyl Isosorbide | 3.00% |
| Phase B Ingredients | |
| DI Water | 74.22% |
| Phenoxyethanol | 0.1% |
| EDTA disodium salt | 0.1% |
| Hydroxyethylacrylate/sodium acryloyldimethyltaurate | 3.0% |
| copolymer & squalane & polysorbate 60 | |

### EXAMPLE 23

Another non-limiting emulsion suitable for use in accordance with the present disclosure includes:

| Ingredient | Amount |
|---|---|
| Water | 67.6% |
| Steareth 40 | 0.8% |
| Glycerine | 4.0% |
| Benzoyl Peroxide 74% | 7.0% |
| Benzyl benzoate | 10.0% |
| Cyclomethicone | 5.0% |
| Ethylene Diamine tetraacetic acid disodium salt (EDTA) | 0.1% |
| Stearyl alcohol | 4.0% |
| Streareth 2 | 1.5% |

A non-limiting example of a suitable sealer for use in accordance with the above emulsion includes the sealer composition shown below:

Sealer

| | |
|---|---|
| Salicylic acid | 2.0% |
| Eudagrid S100 | 1.0% polyacrylic acid |
| SDA 39C ethanol 200 proof | 50% |
| Hydroxypropylcellulose | 1.0% |
| Acetone | 46.0% |

### EXAMPLE 24

Another non-limiting example of a suitable benzoyl peroxide composition includes:

| Ingredient | Amount |
|---|---|
| Benzoyl Peroxide | 6.25% |
| Benzoyl benzoate | 42.45% |
| Dimethyl isosorbide | 40.00% |
| Vitamin E Acetate | 0.5% |
| BHT | 0.8% |
| Ethoxy diglycol | 10.0% |

A non-limiting example of suitable sealer for use with this benzoyl peroxide composition includes the sealer composition shown below:

Sealer

| | |
|---|---|
| Salicylic acid | 2.0% |
| Eudagrid S100 | 1.0% polyacrylic acid |
| SDA 39C ethanol 200 proof | 50% |
| Hydroxypropylcellulose | 1.0% |
| Acetone | 46.0% |

### EXAMPLE 25

In vitro percutaneous absorption studies were performed at the University of California Irvine (“UCI”) using radiolabeled topical benzoyl peroxide serum applied to human cadaver skin. The following protocol was followed:
Group A: Skin was wiped with a wet gauze and allowed 2 minutes to dry. 15 mg of radiolabeled benzoyl peroxide serum was applied to the skin surface and rubbed on with a rubber spatula and allowed 2 minutes to dry.
Group B: Skin was wiped with a wet gauze and allowed 2 minutes to dry. 15 mg of radiolabeled benzoyl peroxide serum was applied to the skin surface and rubbed on with a rubber spatula and allowed 2 minutes to dry. Sealer was applied and allowed to dry and form film.
Group C: Skin was wiped with a wet gauze and allowed 2 minutes to dry. 15 mg of radiolabeled benzoyl peroxide emulsion was applied to the skin surface and rubbed on with a rubber spatula and allowed 2 minutes to dry.
Results: After eight hours of application skin from Group B that utilized benzoyl peroxide serum with the sealer in accordance with the present disclosure provided 23.6% of the benzoyl peroxide applied to the stratum corneum and the epidermis. Skin from Group A, where the benzoyl peroxide serum formula was applied without the sealer enhancement provided 17.5% of the benzoyl peroxide applied to the stratum corneum and the epidermis. The control benzoyl peroxide emulsion (Group C skin) provided 10% of the benzoyl peroxide applied.

The results further showed that the benzoyl peroxide serums (where benzoyl peroxide is dissolved in a solution in Groups A and B) leave lesser amounts of product to be wiped off after eight hours than the emulsions (Group C). This finding was more dramatic when the sealer enhancement was applied on top of the serum (Group B).

Groups A and B left a greater amount of benzoyl peroxide on the surface of the stratum corneum than the emulsion of Group C.

In Group B, the treatment not only delivered the product to the skin in need thereof, but provided a reservoir of product readily available from the remaining serum.

The application of the sealer in Group B greatly improved the penetration of the benzoyl peroxide into the stratum corneum, epidermis and dermis over the compositions applied without a sealer.

### EXAMPLE 26

The benzoyl peroxide composition in the table below is applied to the skin of a person having acne. The sealer composition shown below is immediately applied to the skin where the benzoyl peroxide composition is applied. The sealer forms a barrier over the treated skin preventing the benzoyl peroxide solution from being wiped off of the skin.

| Ingredients | Amount |
|---|---|
| Benzoyl Peroxide Composition | |
| Water | 67.6% |
| Steareth 40 | 0.8% |
| Glycerine | 4.0% |
| Benzoyl Peroxide 74% | 7.0% |
| Benzyl benzoate | 10.0% |
| Cyclomethicone | 5.0% |
| Ethylene Diamine tetraacetic acid disodium salt | 0.1% |
| (EDTA) | |
| Stearyl alcohol | 4.0% |
| Streareth 2 | 1.5% |
| Sealer | |
| Salicylic acid | 2.0% |
| Eudagrid S100 | 1.0% |
| | (polyacrylic acid) |
| SDA 39C ethanol 200 proof | 50% |
| Hydroxypropylcellulose | 1.0% |
| Acetone | 46.0% |

### EXAMPLE 27

The benzoyl peroxide composition shown below in the table is applied to the skin of a person having acne. The sealer composition shown below is immediately applied to the skin where the benzoyl peroxide composition is applied. The sealer forms a barrier over the treated skin preventing the benzoyl peroxide solution from being wiped off of the skin.

| Ingredient | Amount | RANGE OF AMOUNTS |
|---|---|---|
| Benzoyl Peroxide | 6.25% | 2.0%-15% |
| Benzoyl benzoate | 42.45% | 20%-60% |
| Dimethyl isosorbide | 40.00% | 20%-60% |
| Vitamin E Acetate | 0.5% | 0.0%-5% |
| BHT | 0.8% | 0.0%-5% |
| Ethoxy diglycol | 10.0% | 5%-20% |
| Sealer | | |
| Salicylic acid | 2.0% | 1.0%-15% |
| Eudagrid S100 | 1.0% | 0.5%-10% |
| | polyacrylic acid | |
| SDA 39C ethanol 200 proof | 50% | 30%-70% |
| Hydroxypropylcellulose | 1.0% | 0.1%-10% |
| Acetone | 46.0% | 20%-70% |

### EXAMPLE 28

One suitable sealer for use in accordance with the present disclosure includes:

| Sealer | |
|---|---|
| Salicylic acid | 15.0% |
| Eudagrid S100 | 7.5% polyacrylic acid |
| SDA 40B ethanol 200 proof | 34.5 |
| Acetone | 43.0% |

It will be understood that various modifications may be made to the embodiments disclosed herein. Therefore, the above description should not be construed as limiting, but merely as exemplifications of embodiments. Those skilled in art will envision other modifications within the scope and spirit of the claims appended hereto.

What is claimed:

1. A method comprising topically applying a composition comprising benzoyl peroxide; and applying a sealer over the composition.

2. The method according to claim 1 wherein the benzoyl peroxide is in solution form.

3. The method according to claim 1 wherein the sealer comprises a penetration aid in the amount of about 5% to about 20% by weight of the total composition.

4. The method according to claim 3 wherein the penetration aid is 2-hydroxybenzoic acid.

5. The method according to claim 1 wherein the sealer comprises a solvent and at least one polymer.

6. The method according to claim 5 wherein the polymer is selected from the group consisting of natural polymers, acrylic resins, silicones, celluloses, alkyd resins, carboxyvinyl polymers, vinylpyrrolidone-based polymers, methacrylic acid copolymer, and combinations thereof.

7. The method according to claim 5 wherein the polymer is selected from the group consisting of polyacrylic acid, poly(methyl acrylate), poly-(ethyl acrylate), poly(butyl acrylate), polyacrylamide, poly(N-isopropylacrylamide), ammonium polyacrylate, sodium polyacrylate), crosslinked sodium polyacrylate, polymethacrylic acid, poly(methyl methacrylate, poly-(ethyl methacrylate), poly(butyl methacrylate), polymethacrylamide, sodium methacrylate, acrylic acid-styrene-ammonium methacrylate copolymers, acrylic acid-styrene copolymers, acrylic acid-methacrylamide copolymers, alkyl acrylate-styrene copolymers, alkyl acrylate copolymers, ethyl acrylate-acrylamide-acrylic acid copolymers, ethyl acrylate-butyl acrylate copolymers, ethyl acrylate-ethyl methacrylate copolymers, ethyl acrylate-methyl methacrylate-acrylic acid copolymers, ethyl acrylate-methyl methacrylate copolymers, ethyl acrylate-methacrylic acid copolymers, octyl acrylate-styrene copolymers, octyl acrylate-vinyl acetate copolymers, hydroxypropyl acrylate-butylaminoethyl methacrylate-acrylic acid acrylamide copolymers, butyl acrylate-ethyl hydroxymethacrylate copolymers, butyl acrylate-hydroxymethacrylic acid copolymers, butyl acrylate-methyl methacrylate copolymers, butyl acrylate-methacrylic acid copolymers, butyl acrylate-vinyl acetate copolymers; methyl acrylate-ethyl acrylate copolymers, methyl acrylate-styrene copolymers, methoxyethyl acrylate-hydroxyethyl acrylate-butyl acrylate copolymers, methoxyethyl acrylate-hydroxyethyl acrylate copolymers, acrylic resin alkanolamines, methacrylic acid-styrene copolymers, methacrylic acid-butyl methacrylate copolymers, methacrylic acid-methyl methacrylate copolymers, methyl methacrylate-butyl acrylate-octyl acrylate copolymers, methyl cellulose, ethyl cellulose, cationized cellulose, carboxymethyl cellulose, hydroxyethyl cellulose, hydroxypropyl cellulose, hydroxypropylmethyl cellulose, poly(vinyl methyl ether), vinyl methyl ether-ethyl maleate copolymers, vinyl methyl ether-butyl maleate copolymers, styrene-methylstyrene-indene copolymers, toluenesulfonamide resins, polyamide epichlorohydrin, polyethylene-imine, polyethylene glycol-epichlorohydrin-coconut oil alkylamine-dipropylenetriamine condensates, polyvinyl acetal diester aminoacetate, polyvinyl acetal diethylamino-acetate, poly(dimethylmethylenepieridinium chloride), methoxyethylene-maleic anhydride copolymers, dimethyldiallylammonium chloride-acrylamide copolymers, hydrogenated styrene-methylstyrene-indene copolymers, maleic anhydride-diisobutylene copolymer sodium salts, polyvinyl alcohol, polyvinyl butyrate, polyvinyl chloride, vinyl acetate-crotonic acid copolymers, vinyl acetate-styrene copolymers, butadiene-acrylonitrile copolymers, and combinations thereof.

8. The method according to claim 5 wherein the solvent is selected from the group consisting of short chain alkyl esters, ethers, aldehydes, ketones, alcohols, and combinations thereof.

9. The method according to claim 5 wherein the solvent is selected from the group consisting of acetone, ethanol, and combinations thereof.

10. The method according to claim 1 wherein the sealer comprises a polymer system.

11. The method according to claim 1 wherein the sealer comprises constituents selected from the group consisting of polyacrylic acid, salicylic acid, ethanol, hydroxypropylcellulose, acetone, methacrylic acid copolymer, and combinations thereof.

12. The method according to claim 1 wherein the sealer is characterized as a solvent based polymer solution.

13. The method according to claim 1 wherein the sealer further comprises a penetration aid.

14. The method according to claim 13 wherein the penetration aid is selected from the group consisting of enzymes, keratolytic agents, acids, surfactants, DMSO, 1-dodecylazacycloheptan-2-one, 2-hydroxybenzoic acid, and combinations thereof.

15. The method according to claim 1 further comprising the step of drying the sealer.

16. The method according to claim 1 wherein the formulation comprises an additional active ingredient effective in treating acne.

17. The method according to claim 1 wherein the formulation comprises one or more solvents selected from the group consisting of:
short chain alkyl esters of benzoic acid, benzyl alcohol, salicylic acid, phenol or phathalic acid;
short chain alkyl ethers of benzoic acid, benzyl alcohol, salicylic acid, phenol or phathalic acid;
short chain alkyl aldehydes of benzoic acid, benzyl alcohol, salicylic acid, phenol or phathalic acid;
short chain alkyl ketones of benzoic acid, benzyl alcohol, salicylic acid, phenol or phathalic acid;
short chain alkyl alcohols of benzoic acid, benzyl alcohol, salicylic acid, phenol or phathalic acid;
aryl esters of benzoic acid, benzyl alcohol, salicylic acid, phenol and phthalic acid;
aryl ethers of benzoic acid, benzyl alcohol, salicylic acid, phenol and phthalic acid;
aryl aldehydes of benzoic acid, benzyl alcohol, salicylic acid, phenol and phthalic acid;
aryl ketones of benzoic acid, benzyl alcohol, salicylic acid, phenol and phthalic acid;
aryl alcohols of benzoic acid, benzyl alcohol, salicylic acid, phenol and phthalic acid; and combinations thereof.

18. The method according to claim 1 wherein the formulation comprises one or more solvents selected from the group consisting of: alkyl esters of benzoic acid, alkyl esters of benzyl alcohol, alkyl esters of salicylic acid, alkyl esters of phenol, alkyl esters of phthalic acid, alkyl ethers of benzyl alcohol, alkyl esters of phthalic acid, alkyl ethers of benzyl alcohol, alkyl ethers of phenol, and combinations thereof.

19. The method according to claim 1 wherein the formulation comprises one or more solvents selected from the group consisting of: benzoyl benzoate, benzoyl alcohol, diethyl phthalate, benzoic acid 2-phenyl ethyl ester, methyl salicylate, ethyl salicylate, propyl salicylate, butyl salicylate, ethyl benzoate, methyl benzoate, propyl benzoate, butyl benzoate, dimethyl phthalate, diethyl phthalate, benzyl ethyl ether, benzyl methyl ether, phenetole, phenyl acetone, phenyl ethyl alcohol, phenoxyethanol, phenyl acetaldehyde, ethyl phenyl acetate, phenyl methyl ketone, phenyl acetate, benzyl acetate, benzyl aceto acetate, benzyl formate, benzaldehyde, benzyl alcohol, ethyl benzyl alcohol, salicylaldehyde, benzyl salicylate, phenyl tolyl ketone, phenyl benzoate, phenyl ether, dibenzyl ether, benzyl benzoate, benzoic acid, 2-phenyl ethyl ester, and combinations thereof.

20. The method according to claim 1 wherein the formulation further comprising a secondary solvent.

21. The method according to claim 18 wherein the secondary solvent is selected from the group consisting of ethanol, acetone, dimethyl isosorbide, glycol ethers of C₁ to C₆ alcohols with no greater than 2 moles of ethylene oxide, and combinations thereof.

22. The method according to claim 20 wherein the secondary solvent is selected from the group consisting of glycol ethers of phenol with no greater than 2 moles of ethylene oxide, glycol ethers of methanol with no greater than 2 moles of ethylene oxide, glycol ethers of ethanol with no greater than 2 moles of ethylene oxide, glycol ethers of propanol with no greater than 2 moles of ethylene oxide, and combinations thereof.

23. The method of claim 1 wherein the composition is a clear facial serum.

24. The method of claim 1 wherein the composition is a stick.

25. The method according to claim 1 wherein the step of applying a composition further comprises applying a predetermined effective amount of the composition to treat acne.

26. The method according to claim 1, further comprising applying a cleanser to the skin.

27. The method according to claim 1, further comprising applying a toner to the skin.

28. A method of treating acne comprising contacting skin in need of treatment with a topical benzoyl peroxide solution; and applying a sealer to the skin, wherein the sealer comprises a penetration aid.

29. The method of claim 28 wherein the penetration aid is present in an amount of about 5% to about 20% by weight of the total composition.

30. The method according to claim 28 wherein the penetration aid is 2-hydroxybenzoic acid.

31. Use of a penetration aid in the manufacture of a sealer composition for treatment of an acne Vulgaris.

32. The use in accordance with claim 31 wherein the penetration aid is 2-hydroxybenzoic acid.

33. The use in accordance with claim 31 wherein the sealer comprises a penetration aid in the amount of about 5% to about 20% of the total composition.
